(19) United States
(12) Patent Application Publication
(10) Pub. No.: US 2016/0007541 A1
(43) Pub. Date: Jan. 14, 2016

(54) Increasing Harvest (Yield) of Crop Plants Utilizing Thermodynamic Laws on a Whole Plant Basis to Detect Optimal Periods for Exothermic Energy Versus Endothermic Energy Needs

(71) Applicant: Stoller Enterprises, Inc., Houston, TX (US)

(72) Inventors: Jerry Stoller, Houston, TX (US); Albert Liptay, Houston, TX (US)

(21) Appl. No.: 14/797,962

(22) Filed: Jul. 13, 2015

Related U.S. Application Data
(60) Provisional application No. 62/023,632, filed on Jul. 11, 2014.

(51) Int. Cl. A01G 1/00; G01N 33/00; G01N 25/00

(52) U.S. Cl. CPC A01G 1/001; G01N 25/00; G01N 33/0098

(57) ABSTRACT

The present invention relates to the exogenous application of signaling chemicals, such as minerals and/or small signaling molecules, to change the delta T in differing tissues of a plant, such as a crop plant, to increase development and/or productivity of the plant.

### CROSS-REFERENCE TO RELATED APPLICATIONS

This application claims the benefit under 35 U.S.C. 119(e) of U.S. provisional patent application No. 62/023,632 filed Jul. 11, 2014, the contents of which are incorporated herein by reference.

### BACKGROUND OF THE INVENTION

1. Field of the Invention

This invention relates generally to a method for increasing plant productivity, such as crop plant yields, utilizing thermodynamic principles in biological organisms based on the greatest temperature difference (delta T) between sources of energy and sites where the energy is needed. Specifically, the present method relies on the detection of optimal periods when exothermic energy (i.e., high energy potential use) versus endothermic energy needs are present to build increased infrastructure from the germinating seed to the reproductive stages of growth and beyond to harvest. These optimal periods are identified in order to determine the optimal time(s) when the plant is receptive to a much higher level of energy than normal (i.e., exothermic energy) for increasing productivity. A plethora of signaling chemicals can be used to increase delta T in the direction of energy flow to develop the succeeding generation of seed or other economic unit to increase the productivity of the plant.

2. Description of the Related Art

Current commercial practices of crop productivity are mainly fertilizer driven using minerals such as nitrogen, phosphorus, potassium and possibly other minerals required in lesser amounts. These practices do not necessarily address the perspective of various crop plant energy needs or biological effect of hormones, signaling molecules, specific minerals or other entities. Furthermore current production practices may include the use of various pesticides or amendments to address mostly biotic perturbations.

### BRIEF SUMMARY OF THE INVENTION

The exogenous application of signaling chemicals, such as signaling molecules, hormones or even minerals, to plant tissue during specific times of demand for exothermic energy substantially increases productivity of the plant and increases crop production. To determine the optimum period for exogenous application of the signaling molecules, one must understand the temperature differences (delta T's) between different tissues in a plant so that energy from tissues containing higher energy may flow and be directed to plant tissues with lower energy but in need of more energy. For example, during seed germination the ability to increase energy transfer from the storage tissues to a “growth” need in the growing radicle will increase plant productivity. Additionally, the ability to maximize the energy transfer from the mother plant to the child (seed, fruit and/or flower) will increase plant productivity.

While increasing energy flow from the plant tissues containing higher energy to plant tissues with lower energy is important, it is also important to prevent energy flow back to the mother plant, a very common conundrum wherein a lot of crop productivity can be lost. Thus as important as delta T's are, the present method also uses the exogenous application of signaling chemicals to prevent or reduce the reverse flow of energy back to the mother plant from the developing embryos. Applicant has identified specific signaling chemicals which augment energy transfer during times when the plant is receptive to levels of higher energy (i.e. exothermic energy).

### BRIEF DESCRIPTION OF SEVERAL VIEWS OF THE DRAWINGS

The features and advantages of the present invention will become apparent from the following detailed description of a preferred embodiment thereof, taken in conjunction with the accompanying drawings, in which:

FIG. 1 is a graph illustrating energy flow through the measurement of temperature differences (delta T) from higher temperatures in the endosperm (storage of carbohydrates and proteins) and the scutellum (storage of lipids—a very readily available energy right next to the seed embryo), into the rapidly expanding new root system.

FIG. 2 is a graph illustrating temperature differences (delta T) indicating a flow of energy from the temporary storage system in the stalks (stems) of the corn plant to the developing kernels (seeds) on the ear of the corn plant.

FIG. 3 is a graph illustration temperature differences (delta T) wherein the temperature is higher in the kernel developing on the ear of a corn plant than in the cob. The net result is a “reverse” flow of energy from the developing seed to the mother corn plant.

FIG. 4 shows two photos of plant roots at harvest, one without the intervention as provided in Table 2 on the left, about 4 weeks before harvest wherein the dry weight of the roots at harvest are about 14 grams whereas the dry weight of the treated plant roots (photo on the right) was about 23 grams.

FIG. 5 shows a soybean plant wherein the left photo shows a plant that was treated with only the major minerals fertility (nitrogen, phosphorous and potassium) whereas the plant on the left was also treated with a solution of boron (9 wt %) at a rate of 1 pint/acre in furrow at the time of planting (a product known as NITRATE BALANCER™ produced by Stoller USA). The plant was treated just before its flowering period, a time with potentially a lot of new cell division for development of flowers etc. and therefore a time when the plant can use a lot of exothermic energy.

FIG. 6 is a graph showing a rather large delta T just before flowering with foliar application of the 9 wt % boron product used in FIG. 5, with energy flow from the temporary energy storage in the stalk of the corn plant to the vascular system that distributes energy throughout the whole plant as well as to the developing seeds.

### DETAILED DESCRIPTION OF THE INVENTION

Thermodynamically, two types of energy are used in biological development of a crop plant: exothermic energy and endothermic energy. Exothermic energy reflects a rather large, very precise time in plant development when a rather high level of energy is accepted by a plant, mostly to support a high level of new cell formation. These new cells can be synthesized into many specialty cells at a much more leisurely and slower rate than the “flushes” of high energy used by exothermic energy. The much slower rate of plant tissue development is via endothermic energy, when very specialized cells are formed to develop an array of tissues that are required for a developing plant.

It has been surprisingly and unexpectedly found that when the high levels of exothermic energy are accepted by the developing plant through cognitive choice of signaling chemicals that signal for higher energy, the developing plant's productivity can be much more greatly enhanced than is thought to be normal.

The present invention is directed to a method for increasing the productivity of a plant by exogenously applying at least one signaling chemical to the plant at a specific time during at least one growth stage of the plant to specifically target an increase in a temperature difference (delta T) in the direction of energy flow between a first tissue portion of the plant and a second tissue portion of a plant that needs additional energy in order to increase the productivity of the plant. This specific targeting is construed to mean that the delta Ts have been measured and are known by measuring delta Ts of the different tissue portions of a similar plant. This information with respect to the delta Ts is then used in future exogenous application of at least one signaling chemical to similar plants. The method for determining the energy needs of a plant includes the steps of: a) measuring a temperature difference (delta T) between a first tissue portion and a second tissue portion of the plant over time during at least one growth stage of the plant; b) identifying when said delta T reverses or decreases to determine the energy needs of a plant. This information is then used in the future exogenous application of at least one signaling chemical in such plants.

Thermodynamics is the conveyor of energy from a higher source to a lower source of energy. The present invention is directed to a method for providing a signal to the plant by the application of signaling chemicals as shown in the attached series of drawings and tables at specific times of plant growth. Ideally, the exogenous application of a signaling chemical can produce a rather large increase in productivity. Depending on the receptor molecules, the signaling chemical (e.g., mineral(s), hormone(s), signaling molecule(s) or other entity), may give a huge energy increase.

Energy transfer is from matter from one source to another site wherein the greatest multiplicity of particles tends toward high energy level of the particles of matter within the boundaries of increased entropy. The source of the matter of energy can be photonic, soil derived, entity driven, and even from biotic systems. There are two main deltas (temperature and pressure) that delineate where and how energy is transferred. In this invention it is understood that delta pressure is of import. However, the present invention focuses on energy transfer utilizing information related to delta “T's”, for detecting and thence modifying for greater production efficiency.

It is understood that at least two types of roots are synthesized by plants, the small feeder roots close to the soil surface and the larger, deeper roots apparently used for anchoring the plant as well as for storage of photosynthates. The tips of the lateral roots are the sites where water and minerals are taken up from the soil. The tips are where four of the main hormones, cytokinins, gibberellin, ABA and ethylene, as a minimum are synthesized. The synthesis of the lateral roots is driven by auxin moving down the plant. The lateral tips then synthesize the hormones which drive growth and dictate formation of/and the strength of thermodynamic changes of energy flow within the plant. The present invention makes use of the general signaling mentioned above to determine when exothermic energy can take place in plants and then how these signals can best be amplified for increasing, optimizing and/or maximizing flow of energy from the beginning of germination to just before harvest.

At least one signaling chemical may be applied to a variety of seed and/or plants including, but not limited to, the seeds and/or plants of alfalfa, almonds, apples, asparagus, beans, beets (red), berry crops, broccoli, brussel sprouts, cabbage, carrots, cauliflower, celery, cherries, citrus, clover corn, cotton, cucurbits, grapes, kale, lentils, lettuce, melons, nut crops, onion/garlic, oranges, peaches, peanuts, pears, pecans, peppers, pistachios, plums/prunes, potatoes, radishes, rape (canola), raspberries, soybeans, spinach, strawberries, sugar beets, sunflowers, tobacco, tomatoes, tree fruit, turnips, vine crops, walnuts, vegetable crops, wheat/barley/oats, and house plants (gardenias, carnations, African violets).

While routine experimentation may be used following techniques provided herein, the signaling chemicals may be selected from a variety of known minerals, hormones, signaling molecules and other entities that have been determined to increase the delta T in the tissue of a plant. Signaling molecules operate at the time when the plant is receptive to high energy. High energy receptivity reflects high levels of cell division at very specific short windows of time. Cell division at high levels is usually associated with a burst of energy for the plant growing from seed, or the plant producing offspring, or the offspring in the seed for example, the embryos that are in a state of high cell division. While the amount of the signaling molecules may vary, the amount of exogenous application may be at a rate of 0.5 pints-2 gallons per acre, preferably about 0.5-2 pints per acre, or preferably 1 pint per acre. Signaling molecules may include, but are not limited to, BIO-FORGE® (a N,N′-diformyl urea formulation, see U.S. Pat. Nos. 6,040,273 and 6,448,440, which are incorporated herein by reference), NITRATE BALANCER™ (a boron-containing formulation, see U.S. Pat. No. 5,614,653, which is incorporated herein by reference). Additionally, trehalose and gibberellin are known signaling chemicals that may be exogenously applied in accordance with the present invention. Furthermore, cobalt may be exogenously applied as a signaling chemical in accordance with the present invention.

In one embodiment, BIO-FORGE® (a N,N′-diformyl urea formulation produced by Stoller USA, Houston, Tex.) is used as a signaling molecule within the range of 0.01% to 0.1% solution (wt/wt), preferably at a concentration of about 0.03% solution (wt/wt). Second, a boron-containing solution having a concentration within the range of 0.01 to 1% solution (wt/wt), preferably at a concentration of about 0.03% solution (wt/wt) of about 4-12% boron product (wt/wt), or 8-10% boron product (wt/wt), or about 9% boron product (wt/wt) distinctly is exogenously applied to assist in lowering the temperature of the end delivery point by creating a favorable delta T for energy transfer from a source or through foliar treatment of the plant. The concentration in plant tissue (as routinely detected by commercial labs) is suggested to be >50 ppm, or >55 ppm, or >60 ppm. In another embodiment a divalent solution including iron, nickel, sulfur, manganese and/or zinc may be applied to a plant, during the reproductive growth stages. The divalent ions drive the necessity for the plant to increase delta P (water evaporation) to thereby control the temperature gradient so that delta T is maintained between plant cells and within plant cells. Thus divalent metals play an important part in the regulation of partitioning of energy within a plant. When both a boron-containing solution and a divalent solution are applied to a plant as provided above, the growth and productivity of the plant is further maximized. In simple terms boron cools that plant tissue and divalent metals increase the temperature of plant tissue. This causes an increase in delta P (evaporation) so that the plant can remain cooler than the environment around it. Thus the plant can absorb more energy. It is also understood that other signaling molecules, such as trehalose, may be exogenously applied to the plant to increase delta T for increased plant productivity

The following examples are provided for illustrative purposes as one of skill in the art would readily understand how to modify the examples within the scope of the present invention. Therefore the present invention is not limited by these examples.

### Examples

### Germination Thermodynamics

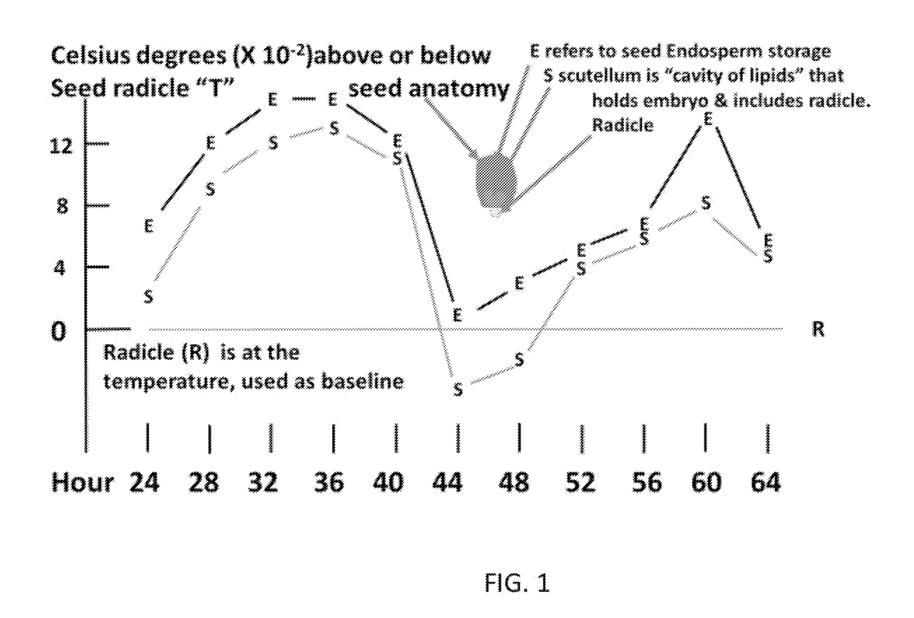

In order to determine the energy that flows through a germinating seed, the temperature difference (delta T) between the emerging radicle (first root) and the storage parts of the seed (scutellum and endosperm) were measured. Thermocouples (very thin wires down to the thickness of a human hair, referred to as “Thysitemp” IT-23 implantable thermocouple microprobes from Physitemp Instruments Inc in Clifton N.J.) were used to collect temperature data on a very accurate and precise datalogger (brand name DATATAKER with accuracy down to 5 decimal points of a degree). This temperature data was gathered and averaged over each hour. The thermocouples were inserted into the radicle (R), scutellum (S) and endosperm (E) by first forming a slight insertion and then inserting the thermocouple microprobes into the minute parts of the seeds. The radicle temperature was used as the base temperature, while the scutellum and endosperm (E) had the higher temperatures as depicted by FIG. 1. The data in FIG. 1 reflect the movement of energy from a higher temperature in the storage seed parts, scutellum (S) and endosperm (E), to a lower temperature in the Radicle (R). FIG. 1 illustrates the energy flow through the measure of temperature difference (delta T) between the seed storage organs, endosperm and scutellum, into the quickly expanding radicle in a new root system. As long as the temperature is higher in the scutellum and endosperm than in the radicle, energy flows into the developing new root system. However, if the temperature of the storage organs becomes lower than that of the radicle reversal of energy flow takes place and the storage organs take energy from the new developing root. This is a conundrum that is solved by the present invention to prevent and/or reduce the energy lost to the mother plant.

Table 1 illustrates that this high energy acceptance by the developing roots is what contributes to development of a strong and effective root system. What is fascinating is that with a signaling molecule, such as BIO-FORGE®, a huge increase in overall life-long crop productivity can be garnered at this point by an “In Furrow” treatment with BIO-FORGE® at a rate of from 0.1 pint per acre to 2 pints per acre, preferably 1 pint per acre. The data in Table 1 confirms that a thermodynamically higher energy episode is present during the plethora of growth stages of the plant wherein a rather high level of energy can be utilized by the plant, then a signaling molecule such as BIO-FORGE® can signal to the plant to use a near maximum amount of potential energy for new cell division.

TABLE 1

| | Yield | | | Seed size |
|---|---|---|---|---|
| | (bushels | Yield t test | 1,000 seed | t test |
| Treatment | per acre) | 1 vs 2 | weight (g) | 1 vs 2 |
| 1. Control | 155 bu | | 302 | |
| 2. BIO-FORGE ® | 302 bu | P = <1% | | P = <1% |
| applied in the | | Highly | | Highly |
| furrow at the time | | significant | | significant |
| of seed sowing | | | | |

With this understanding, BIO-FORGE®, a signaling molecule comprised of di-formyl urea (supplied by Stoller USA, Houston Tex.) was applied right at the beginning of seed sowing just before there was a large difference in temperature (delta T) between the seed storage organs and the radicle (root). As seen in Table 1, the result was a continuing signal during the whole growth of the corn crop that showed a huge doubling of yield from 155 bushels per acre of corn to 302 bushels of corn per acre.

It was not known prior to this invention when the first burst of higher energy (exothermic) would be accepted by the developing root. Surprisingly, this acceptance of high energy occurs well before any visual observance of even any significance emergence of the radicle root (first root) at between 0 and 44 hour after watering the seed. This acceptance of higher energy is strictly relegated to this time. If this window of time is not taken advantage of, then this rather very large yield potential through exothermic energy is lost. The plant simply cannot accept a higher energy level during the slower and more complicated phases of growth whilst endothermic energy develops the various plethora of cell types (cell differentiation). Serious debilitating effects of disturbing slower endothermic growth can ensue.

Energy Flow from Mother to Seed

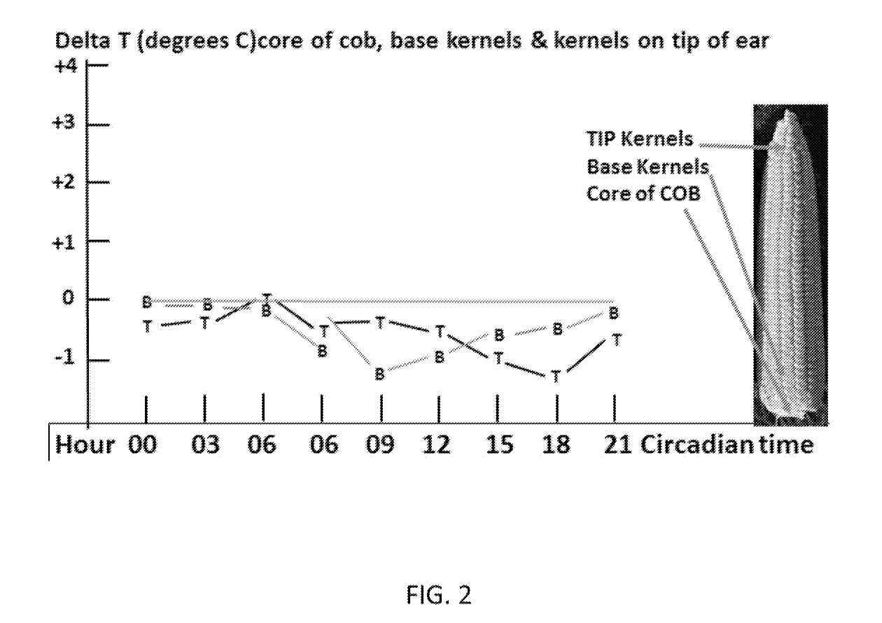

Using similar testing techniques and equipment as provided above, temperature measurements were taken at the core of a cob (baseline) and at the Tip Kernels (T) and Base Kernels (B) to determine the temperature difference (delta T) and hence the flow of energy in an ear of corn. FIG. 2 illustrates that at air temperature <30 C it is generally acceptable for positive flow of energy (delta T) from the mother plant to the developing seed kernels on the corn ear. The energy flows from the inside of the ear (cob) which is directly associated with the temporary energy storage system of the corn plant. It is estimated that if the actual temperatures are less than 30 degrees Celsius, then there is a fair chance that the delta T indicates energy flows from a storage source in the mother plant and into the seeds developing on the ear of corn plant.

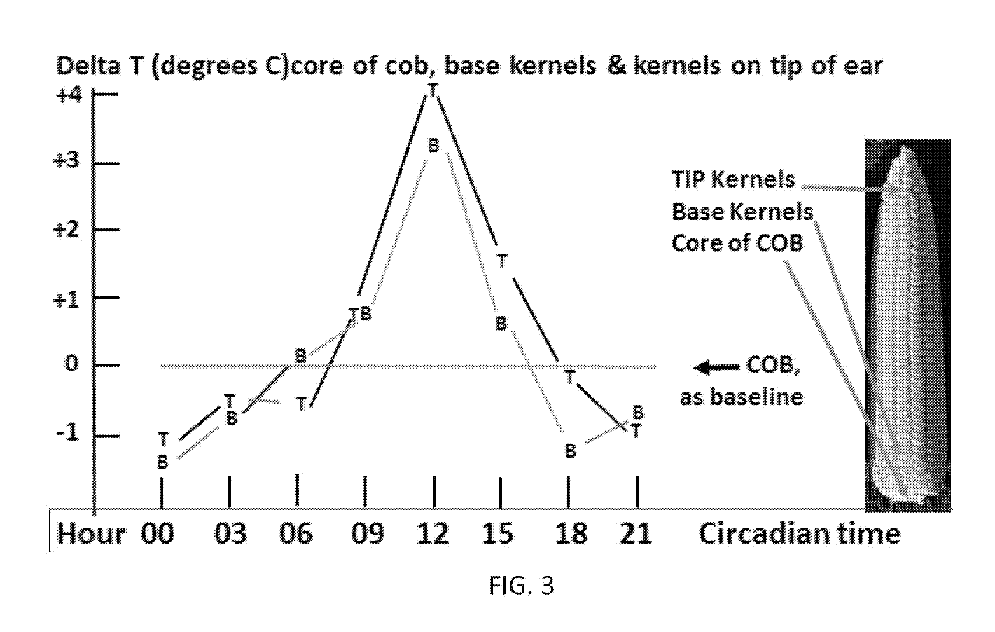

FIG. 3 is a graph illustration temperature differences (delta T) wherein the temperature is higher in the kernel developing on the ear of a corn plant than in the cob. The net result is a “reverse” flow of energy from the developing seed to the mother corn plant. This reverse flow appears to take place when outside temperatures are greater than 30 degrees Celsius. This can often be a huge problem in many crops including soybeans. In the case of this rather extreme change in delta T, both in direction of flow of energy but also in the magnitude of the change represent big losses usually that are not so easily “seen” but are real.

It was unexpectedly and surprising found that a signaling molecule could arrest this reverse flow of energy to the mother plant. Not only was the reverse flow halted but new and sufficient energy was synthesized by the mother plant to “look after” itself as well as the developing seed kernels on the ear. For example, it was found that this loss can be mitigated by the exogenous application of the signaling molecule, “trehalose”, FORCE™ (produced by Stoller USA, Houston, Tex.), combined with gibberellin. The trehalose is used at 0.1-2 pints per acre, preferable at a rate of 1 pint per acre while the gibberellin is used at a pint per acre of 4% gibberellin as a preferred rate but with a range of 0.1 to 10 pints per acre. In one preferred embodiment, the signaling chemicals were applied via a foliar application such that a trehalose concentration of 100 g per acre combined with a gibberellin concentration of 18 g per acre were used.

Table 2 provides data illustrating how the signaling molecule trehalose in concert with the plant hormone gibberellin can mitigate the potential loss caused by reverse energy flow as depicted in FIG. 3. The larger root system after treatment indicates sufficient energy has been formed for the mother plant as well as all the seed. New energy is synthesized for both the needs of the mother plant as well as developing seeds. The results are that nearly a 40 bushel increase of corn seed is produced with this intervention.

TABLE 2

| | Yield | | |
|---|---|---|---|
| | (bushels per | 1,000 seed | Dry weight |
| Treatment | acre) | weight (g) | roots (g) |
| 1. Control | 156 | 323 | 14.5 grams |
| 2. At the R2 stage of | 188 | 345 | 23.7 grams |
| corn growth, apply | | | |
| trehalose foliar with | | | |
| gibberellin as shown | | | |
| in FIG. 3 | | | |
| | T test p = 0.01 | T test | T test |
| | Highly | P = 0.01 | P = 0.01 |
| | significant | Highly significant | Highly |
| | | | significant |

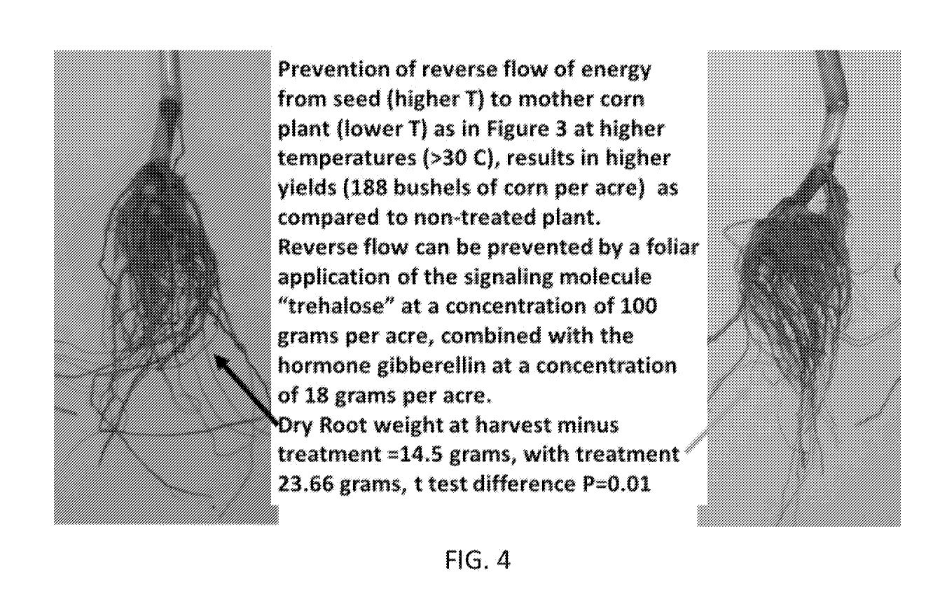

FIG. 4 illustrates the larger root system of the treated plants (right) indicating sufficient energy for redevelopment of the mother plant's root system as well as the general well-being of the mother plant and the seed kernels. FIG. 4 shows 2 photos of plant roots at harvest, one without the intervention of Table 2 on the left, about 4 weeks before harvest wherein the dry weight of the roots at harvest are about 14 grams whereas the dry weight of the treated plant roots (photo on the right) was about 23 grams. Thus not only did the process of the invention stop reverse energy flow but the roots of the corn plants synthesized about the weight equivalent of 60% of the weight of the former roots. All of the plant was also more energy rich.

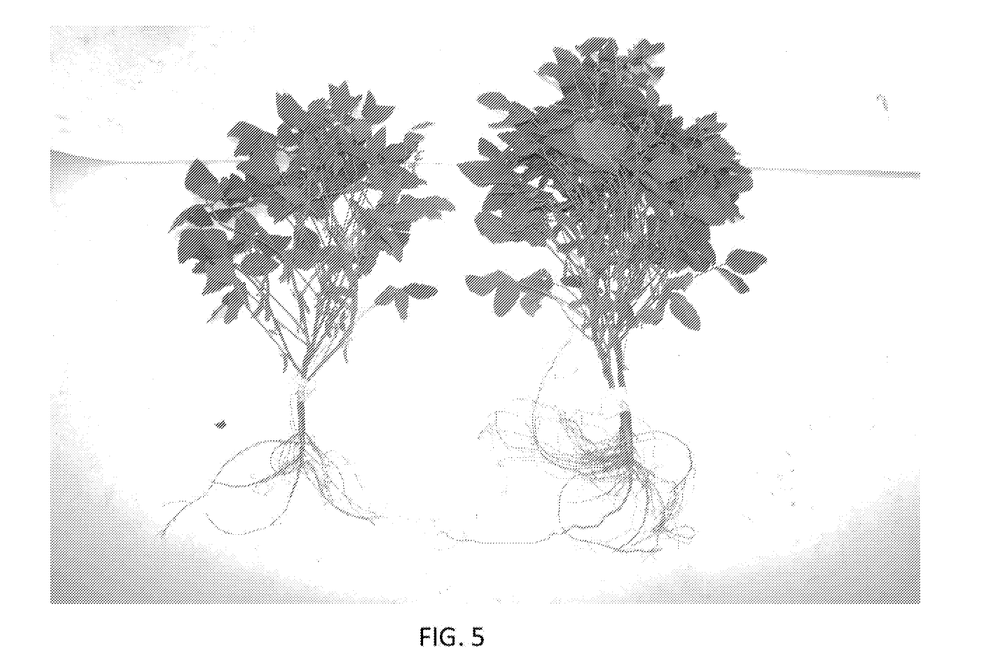

FIG. 5 shows a soybean plant treated with a product, NITRATE BALANCER™ (sold by Stoller USA, Houston, Tex.) having a boron content of 9% (wt/wt). The plant was treated just before its flowering period, a time with potentially a lot of new cell division for development of flowers etc. and therefore a time when the plant can use a lot of exothermic energy. The plant was a shorter, stalkier plant and therefore a more productive plant, with bigger roots, many more branches and therefore more pods and soybean seeds.

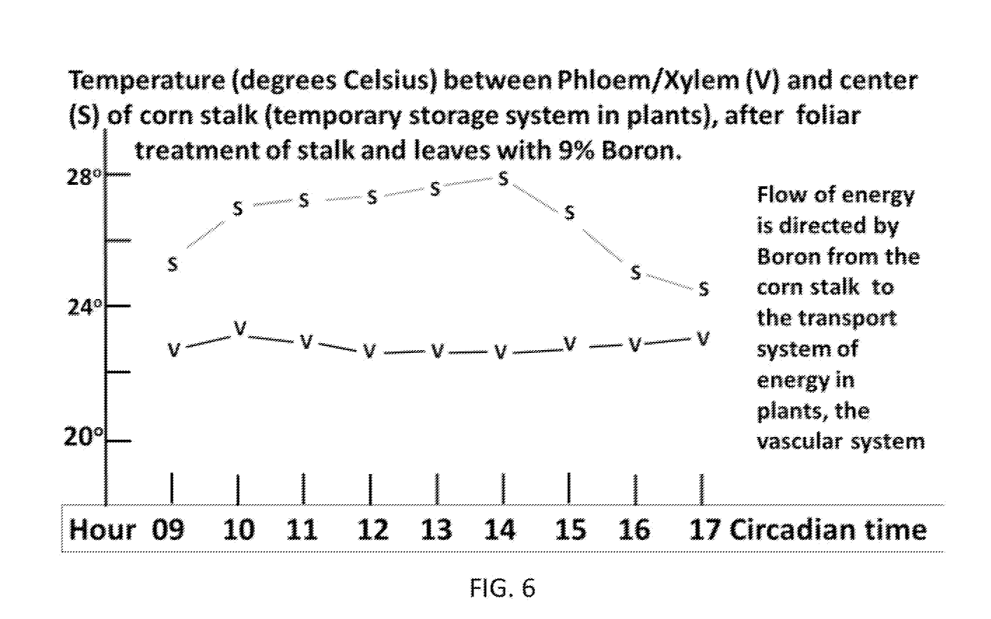

FIG. 6 illustrates the power of the mineral boron in lowering the temperature of the “energy receiving” tissue thus directing energy flow to the site needing the energy. Boron was applied via exogenous foliar application using NITRATE BALANCER™ (sold by Stoller USA, Houston, Tex.) which contains boron at 9%. A pint per acre is the preferred rate but also within the range of 0.1 to 5 pint per acre. Note the massive delta T wrought by boron. The energy is directed from the stalk to the vascular transport system for overall distribution of energy to the whole plant including the developing seeds. The treatment makes a shorter stalkier plant with a better root system, more branching as well as better seed development.

Table 3 illustrates the benefits of the exogenous application of boron lowering the temperature of the receiving site for energy thus greater flow of energy caused by the use of boron as in FIG. 5. Table 3 are data indicating a doubling of yield after application of the boron product to lower the temperature of the developing root system in early germination, and thereby having a delta T transferring more energy to the developing new corn root.

TABLE 3

| | Yield (bushels per | | |
|---|---|---|---|
| Treatment | acrea) | Yield increase | T test |
| 1. Control | 50.7 bu | | |
| 2. Boron at a | 105.3 bu | 107% | P = 0.01 |
| pint per acre | | | Highly significant |
| of nitrate | | | |
| balancer with | | | |
| 9% boron | | | |
| 3. Boron applied | 124.7 | 146% | |
| as a foliar (to | | | |
| leaves | | | |
| between V1 | | | |
| and V2 stage | | | |
| of growth) | | | |

Table 4 shows data of the effect of the mineral cobalt applied into the soil via a drip irrigation system for tomato plants. The base fertility in contrast to the addition of cobalt resulted in a significant increase in yield reflecting a higher energy mediated by cobalt. Not only was yield significantly increased but the taste of the tomatoes was greatly improved and the percent sugars were increased as well.

TABLE 4

| | Yield (number | | | |
|---|---|---|---|---|
| | of tomato fruits | | | Taste test 14 |
| | per acre × | Yield (No. of | % Brix | scientific |
| Treatment | 1,000) | tons per acre) | (sugars) | personnel |
| 1. Base | 244 | 29.6 | 4.8 | 2 |
| with | | | | |
| fertility | | | | |
| 2. As # 1 | 288 | 33.9 | 5.5 | 2.8 |
| and with | | | | |
| cobalt @ | | | | |
| 1 pt/acre | | | | |
| over 5 | | | | |
| weeks | | | | |
| | T test p = 0.05 | T test p = 0.05 | P = 0.05 | P = 0.01 |
| | Significant | Significant | Significant | Highly |
| | | | | significant |
| | | | | improvement in |
| | | | | taste |

Although the present invention has been disclosed in terms of a preferred embodiment, it will be understood that numerous additional modifications and variations could be made thereto without departing from the scope of the invention as defined by the following claims:

1. A method for increasing the productivity of a plant comprising the step of:
exogenously applying at least one signaling chemical to the plant at a specific time during at least one growth stage of the plant to specifically target an increase in a temperature difference (delta T) in the direction of energy flow between a first tissue portion of the plant and a second tissue portion of a plant that needs additional energy in order to increase the productivity of the plant.

2. The method of claim 1 wherein said at least one signaling chemical is at least one chemical selected from the group consisting of signaling molecules, hormones, minerals, and combinations thereof.

3. The method of claim 1 wherein said at least one signaling chemical is selecting from the group consisting of N,N′-diformyl urea, boron, iron, nickel, sulfur, manganese, zinc, trehalose, gibberellin, cobalt and combinations thereof.

4. The method of claim 1 wherein said at least one signaling chemical is exogenously applied to a seed during germination.

5. The method of claim 4 wherein said at least one signaling chemical is exogenously applied to said seed in furrow.

6. The method of claim 5 wherein said at least on signaling chemical is exogenously applied to said seed within 44 hours after the beginning of germination.

7. The method of claim 6 wherein said seed is a corn seed.

8. The method of claim 1 wherein said plant is selected from the group consisting of corn, soybean, and tomato.

9. The method of claim 1 wherein said signaling chemical is N,N-diformyl urea applied exogenously via an in furrow application.

10. The method of claim 9, wherein said signaling chemical is N,N-diformyl urea applied as a solution having a concentration of 0.01-0.1 wt % N,N-diformyl urea.

11. The method of claim 10, wherein said solution is applied at a rate of from 0.1-2 pints per acre.

12. The method of claim 1, wherein said signaling chemical is boron applied exogenously via an in furrow application.

13. The method of claim 1, wherein said at least one signaling chemical is exogenously applied as a foliar application.

14. The method of claim 13, wherein said at least one signaling chemical is a combination of trehalose and gibberellin.

15. The method of claim 13, wherein said trehalose is exogenously applied at a rate of 0.1-2 pints per acre and said gibberellin is exogenously applied at a rate of 0.1 to 10 pints per acre.

16. The method of claim 13, wherein said at least one signaling chemical is applied at the R2 stage of growth of the plant.

17. The method of claim 13, wherein said at least one signaling chemical is boron.

18. The method of claim 17, wherein boron is exogenously applied during the vegetative stage of growth between stage V1 and V2.

19. The method of claim 1, wherein said at least one signaling molecule is cobalt applied to soil via a drip irrigation system.

20. The method of claim 1, wherein said cobalt is exogenously applies at a rate of about 1 pint/acres over 5 weeks.

21. The method for determining the energy needs of a plant comprising the steps of:
a) measuring a temperature difference (delta T) between a first tissue portion and a second tissue portion of the plant over time during at least one growth stage of the plant;
b) identifying when said delta T reverses or decreases to determine the energy needs of a plant.

22. The method of claim 21 wherein said temperature difference are measured using thermocouples inserted into the first tissue portion and the second tissue portion.

23. The method of claim 22, wherein said thermocouples are able to measure down to 5 decimal points of a degree of Celsius.

24. The method of claim 23, wherein said first tissue portion is a storage part of a seed and said second tissue portion is an emerging radicle of said seed.

25. The method of claim 24, wherein said first tissue portion is the scutellum or the endosperm, or both.

26. The method of claim 23, wherein said first tissue portion is the inside of a cob of a corn plant and said second tissue portion is a kernel of said corn plant.

27. The method of claim 26, wherein said second tissue portion is a tip kernel, a base kernel, or both.

28. The method of claim 23, wherein said first tissue portion is the phloem/xylem and said second tissue portion is a corn stalk.
